(19) United States
(12) Patent Application Publication
(10) Pub. No.: US 2026/0056163 A1
(43) Pub. Date: Feb. 26, 2026

(54) PRINTED MULTI-ANALYTE DISPOSABLE ELECTRONIC SENSOR STRIP

(71) Applicant: THE REGENTS OF THE UNIVERSITY OF COLORADO, A BODY CORPORATE, Denver, CO (US)

(72) Inventors: Elliot J. STRAND, Boulder, CO (US); Gregory L. WHITING, Boulder, CO (US)

(21) Appl. No.: 19/104,752

(22) PCT Filed: Aug. 25, 2023
(86) PCT No.: PCT/US2023/072886

§ 371 Date: Feb. 19, 2025

Related U.S. Application Data
(60) Provisional application No. 63/401,299, filed on Aug. 26, 2022.

(51) Int. Cl. G01N 27/414; G01N 27/30; G01N 33/24

(52) U.S. Cl. CPC G01N 27/414; G01N 27/301; G01N 27/302; G01N 33/245

(57) ABSTRACT

Disclosed are systems and methods for measuring data from an environment. The system may be an environmental sensor that includes a substrate and four layers. The first layer may include source and drain electrodes. The second layer may include at least three electrodes including a working electrode that is a gate material comprising a blend of carbon ink and an indicator and a Ag/AgCl reference electrode. The third layer may include a semiconducting channel formed of PEDOT:PSS. The fourth layer may include a dielectric ink. The environment sensor may be used to determine an electrical conductivity, calculate a temperature, and measure a pH of the environment.

### CROSS-REFERENCE TO RELATED APPLICATIONS

This application claims priority to, and the benefit of, U.S. Provisional Application Ser. No. 63/401,299, filed Aug. 26, 2022, and entitled “PRINTED MULTI ANALYTE DISPOSABLE ELECTRONIC SENSOR STRIP,” the entire contents of which are incorporated by reference herein in their entireties.

### GOVERNMENT RIGHTS

This invention was made with government support under grant number 1935594 awarded by the National Science Foundation. The government has certain rights in the invention.

### BACKGROUND

In 2017, the United States spent $359.8 billion on farm production. The two largest expenditures of crop farms were combined crop input (chemicals, fertilizers, and seeds, etc.) and labor, which account for 28.2% and 13.8% of the total expenditure, respectively. The expenditure on labor has been increasingly reduced by mechanization; however, in gaining labor efficiency by moving to large-scale uniform farming, farmers sacrificed resource efficiency due to the heterogeneity of farmlands. To further reduce the cost of large-scale farming by tailoring soil and crop management in heterogeneous fields, precision agriculture (PA) technologies have been widely adopted since the early 1990s. Modern PA relies heavily on the temporal and site-specific environment, soil, and crop data collected by various sensors.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Disclosed embodiments include an environmental sensor for collecting data in an environment. The environmental sensor comprises a substrate and four layers including contacts, gate electrodes, a semiconducting channel, and a dielectric encapsulant.

In some aspects, the techniques described herein relate to an environmental sensor, including: a substrate; a first layer including at least one source electrode and at least one drain electrode; a second layer including at least three electrodes; a third layer including a semiconducting channel; and a fourth layer including a dielectric ink.

In some aspects, the techniques described herein relate to a method for gathering data from an environmental sensor, including: providing an environmental sensor, wherein the environmental sensor includes a substrate, a silver contact, gate electrodes, a semiconducting channel, and a dielectric encapsulant; implanting the environmental substrate into an environment; determining an electrical conductivity of the environment; calculating a temperature of the environment; and measuring a pH of the environment.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings described below.

FIG. 1 illustrates an example of an exploded view of an environmental sensor.

FIG. 2 illustrates an example of an environmental sensor in an environment.

FIG. 3 illustrates a flow chart of an example method of gathering data from an environment.

FIG. 4 illustrates example experimental results comparing a commercial sensor to an example disclosed embodiment of an environmental sensor.

### DETAILED DESCRIPTION

Disclosed embodiments include an environment sensor platform that provides unique readings from a single sensor. The environmental sensor may comprise a substrate and four layers screen printed on the substrate. Each layer may be used to sense different data from an environment. For example, the environmental sensor may comprise a pH sensitive gate material. The pH sensitive gate material may comprise a blend of carbon ink and an indicator. In at least one embodiment, this sensor can detect pH by using the gate material as the working electrode, the channel material as the counter electrode, and the Ag/AgCl gate as the reference electrode. Additionally, the environmental sensor may comprise a semiconducting channel formed of poly(e,4-ethylenedioxythiopene) polystyrene sulfonate (PEDOT:PSS). In an embodiment, the environmental sensor further includes a water sensing element, source and drain electrodes, and an encapsulation layer.

### Example Environmental Sensor

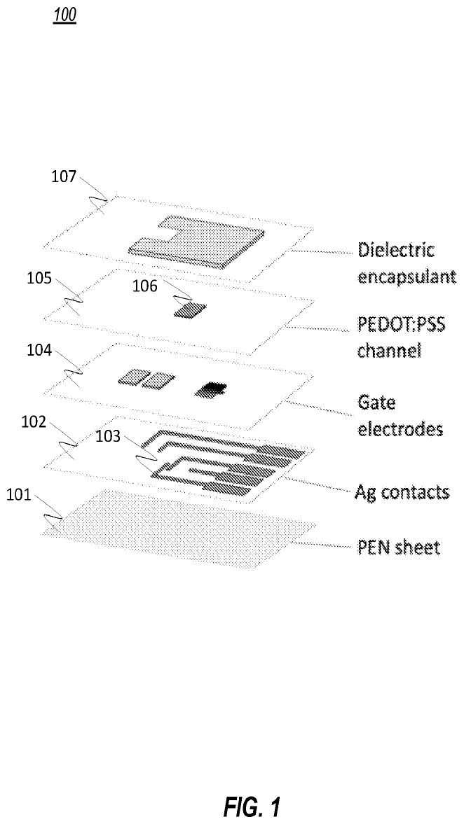

FIG. 1 illustrates an example embodiment of an environmental sensor 100 in an exploded view. As shown, the environmental sensor includes a substrate 101. In embodiments, the substrate 101 may be reasonably flat and have an adequate surface energy. In some embodiments, the substrate is biodegradable, compostable, and/or recyclable. In some embodiments, the substrate is formed of flexible polyethylene naphthalate (PEN). In other embodiments, the substrate may be formed of other materials such as PHBV, PLA, PCL, PBAT, Cellulose Acetate, Balsa wood, or other appropriate biodegradable, flexible materials. In at least one embodiment, 99.5% of the mass of an environmental sensor is the substrate material.

Additionally, FIG. 1 illustrates four printed layers on the substrate 101. In some embodiments, the layers are screen printed onto the substrate using a screen printing process. In some embodiments, the screen printing process utilizes stainless-steel mesh screens.

The first layer 102 includes source and drain electrodes. In some embodiments, there may be one source electrode, two source electrodes, three source electrodes, or more than three source electrodes. Similarly, in some embodiments, there may be one drain electrode, two drain electrodes, three drain electrodes, or more than three drain electrodes. In some embodiments, the number of source and drain electrodes are equal. In other embodiments, the environmental sensor may include more source electrodes or more drain electrodes. In embodiments, the first layer is printed using a silver (Ag) ink creating Ag contacts 103 on the first layer.

The second layer 104 includes gate electrodes. In some embodiments, and as shown in FIG. 1, the environmental sensor 100 includes three gate electrodes. In embodiments, one of the gate electrodes may be a working electrode, another electrode may be a reference electrode, and the third gate electrode may be a counter electrode. In embodiments, the reference and counter electrodes may be formed of Ag/AgCl and the working electrode is formed of a functionalized carbon composite ink and an indicator. In some embodiments, the indicator may be alizarin, phenolphthalein, bromocresol green, methyl red, bromocresol purple, chlorophenol red, or other pH indicators. In some embodiments, the indicator may be a mix of two or more indicators.

The third layer 105 includes a semiconducting channel 106. In some embodiments, the semiconducting channel is formed of poly(e,4-ethylenedioxythiopene) polystyrene sulfonate (PEDOT:PSS), which is an electronic/ionic conductor. PEDOT:PSS has high performance, is easy to process and commercially available. In some embodiments, the semiconducting channel may be formed of another electronic/ionic conductor material.

The fourth layer 107 includes a dielectric encapsulant. In some embodiments, the dielectric encapsulant may be formed of a dielectric ink and encapsulates all device surfaces except the gates, channel, and contact pad areas.

The four printed layers include Ag, Ag/AgCl, functionalized C and an indicator, PEDOT:PSS and a dielectric encapsulant. In at least one embodiment, the environmental sensors are utilized for sensing in a controlled environment agriculture (CEA). The detection media may comprise hydroponic fluid, whole plant sap, aquaponic solutions, waste streams, etc. Additionally, in at least one embodiment, a single environmental sensor (e.g., a single printed test strip sensor) can be used to detect electrical conductivity (EC) (total nutrient (ion) content), temperature, and/or pH level (concentration of hydrogen ions by interrogating the environmental sensor using three different techniques.

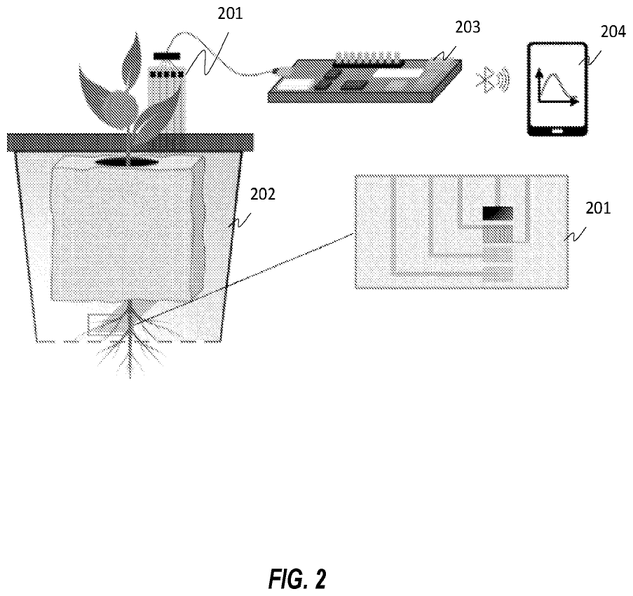

FIG. 2 illustrates an example of an environmental sensor 201 implanted into an environment 202 and communicating with an output sensor 203 (e.g., a potentiostat chip). In some embodiments, the output sensor 203 may include a screen to directly see results from the environmental sensor 201. In some embodiments, and as shown in FIG. 2, the output sensor 203 may communicate with device 204 to visualize results from the environmental sensor 201. The output sensor 203 and the device 204 may be connected via a network, Bluetooth, wired connection, or other appropriate connections. In some embodiments, the environment 202 may be a hydroponic environment. In other embodiments, the environment 202 may be soil, a substrate grow cube, or a greenhouse.

### Example Manufacturing Process of an Example Environmental Sensor

In some embodiments, the environmental sensor 100 is manufactured using a screen printing process using stainless-steel mesh screens. In embodiments, the substrate 101 is rinsed with acetone and isopropanol. In some embodiments, the substrate is rinsed for less than 5 minutes, for about 5 minutes, for about 10 minutes, for about 15 minutes, or for more than 15 minutes.

In embodiments, the first layer 102 is printed with Ag ink. The second layer may be cured at about 50° C., about 75° C., about 100° C., about 125° C., or about 150° C. for about 10 minutes, about 15 minutes, about 30 minutes, or about 45 minutes. The first layer 102 has a final dry thickness of about 15 μm, about 20 μm, about 25 μm, or about 30 μm.

In embodiments, the second layer 104 is printed with Ag/AgCl and C composite inks. The second layer may be cured at about 50° C., about 75° C., about 100° C., about 120° C., or about 150° C. for about 10 minutes, about 15 minutes, about 20 minutes, or about 30 minutes. The Ag/AgCl may have a dry thickness of about 15 μm, about 20 μm, about 25 μm, or about 30 μm. The C composite ink may have a dry thickness of about 5 μm, about 7.5 μm, about 9 μm, or about 10 μm.

In embodiments, the third layer 105 is printed with PEDOT:PSS. The third layer 105 may be annealed at about 50° C., about 75° C., about 100° C., about 120 ° C., or about 150° C. for about 10 minutes, about 15 minutes, about 20 minutes, or about 30 minutes. The third layer 106 may have a final thickness of about 500 nm, about 700 nm, about 800 nm, or about 1000 nm.

In embodiments, the fourth layer 107 is printed with a dielectric ink. The fourth layer 107 may be annealed at about 50° C., about 75° C., about 100° C., about 120° C., or about 150° C. for about 10 minutes, about 15 minutes, about 20 minutes, about 30 minutes, or about 40 minutes. The fourth layer 107 may have a dry thickness of about 20 μm, about 25 μm, about 30 μm, or about 40 μm.

In embodiments, the fourth layer 107 may encapsulate all device surfaces except the gates, channel, and contact pad areas. In some embodiments, the exposed gate areas may have an area of about 5 mm², about 8 mm², about 8.8 mm², about 9 mm², or about 10 mm². The exposed channel area may be about 7 mm², about 8 mm², about 8.2 mm², about 9 mm², or about 10 mm². The interdigitated electrode fingers underneath the channel may have a width of about 100 μm, about 200 μm, about 250 μm, about 300 μm and may overlap the channel by about 200 μm, about 250 μm, or about 300 μm.

### Example Method

The following discussion now refers to a number of methods and method acts. Although the method acts are discussed in specific orders or are illustrated in a flow chart as occurring in a particular order, no order is required unless expressly stated or required because an act is dependent on another act being completed prior to the act being performed.

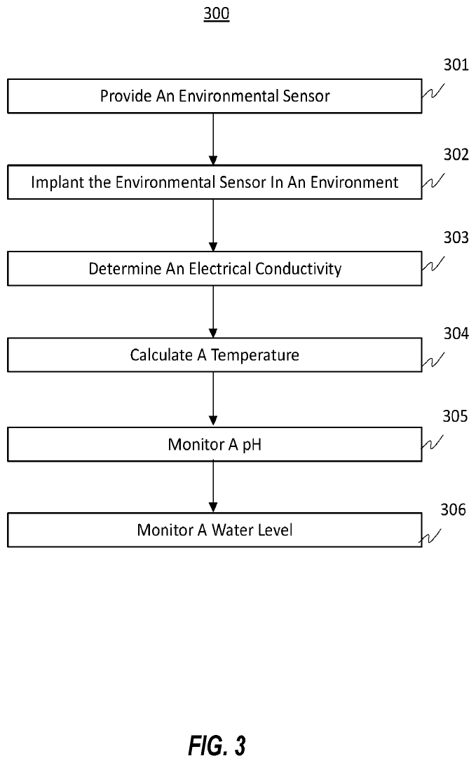

FIG. 3 illustrates a flow chart of an example method for taking measurements of an environment. Referring to act 301, an environmental sensor is provided (e.g., environmental sensor 100). The environmental sensor may include a substrate (e.g., substrate 101), a silver contact (e.g., Ag contacts 103), gate electrodes (e.g., second layer 104), a semiconducting channel (e.g., semiconducting channel 106), and a dielectric encapsulant (e.g., fourth layer 107).

Referring to Act 302, the environmental sensor may be implanted into an environment (e.g., a hydroponic environment). Act 303 includes determining an electrical conductivity of the environment. In some embodiments, the electrical conductivity of the environment is determined by interrogating a transistor potentiostatically and extracting a steady-state current response.

Act 304 includes calculating a temperature of the environment. In some embodiments, the temperature may be calculated by applying a ratio to the EC and a normalized EC. Act 305 includes measuring a pH of the environment. In some embodiments, the pH is monitored by performing a square wave voltammetry (SWV) scan and comparing a peak voltage to a calibration curve.

Optionally, in some embodiments, act 306 includes monitoring a water level of the environment by examining a transistor output characteristic.

### Experimental Results

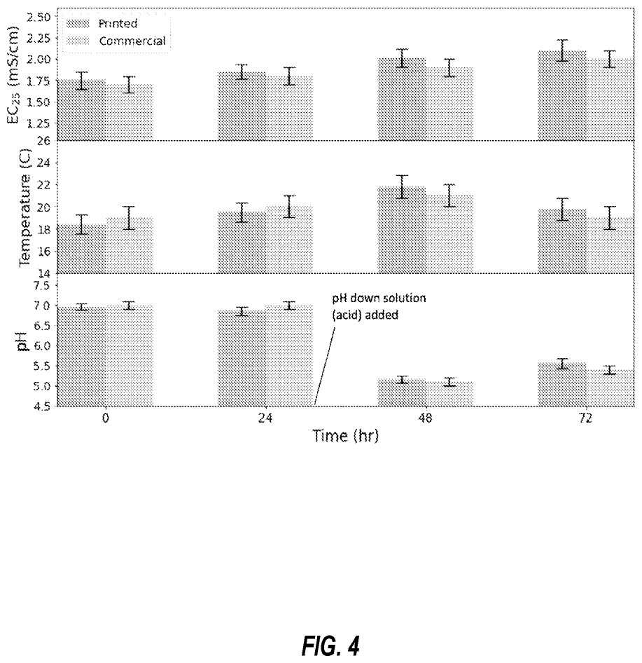

FIG. 4 illustrates comparisons of commercial sensors with an example environmental sensor 100 in a CEA growing environment. The environment was an in-house deep water culture (DWC) hydroponic system and grew tomato (Solanum lycopersicum) plants. The commercial sensors and example environmental sensor was submerged into the chamber to collect real-time hydroponic data. The devices were interrogated once every 24 hours to derive the EC, the temperature, and the pH. As shown in FIG. 4, the environmental sensor was able to accurately monitor EC, temperature, and pH of the active hydroponic fluid over 72 hours.

The increase in solution conductivity is attributed to the tomato plants taking up water which results in a higher concentration of ions in the fluid. The temperature remained consistent between 19° C. and 21° C., which is expected due to the device being interrogated at the same time each day.

Additionally, both the example environmental sensor and the commercial sensor observed a dramatic drop in pH due to the addition of a small volume of a “pH down” hydroponic buffer solution. This buffer solution was added to reach an ideal range for tomato growth.

### Additional Terms

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” “reasonably,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “electrode”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### Example Embodiments

Following are some further example embodiments of the invention. These are presented only by way of example and are not intended to limit the scope of the invention in any way. Further, any example embodiment can be combined with one or more of the example embodiments.
Embodiment 1. An environmental sensor, comprising: a substrate, wherein the substrate is reasonably flat; a first layer including at least one source electrode and at least one drain electrode; a second layer including at least three electrodes; a third layer including a semiconducting channel; and a fourth layer including a dielectric ink.
Embodiment 2. The environmental sensor of embodiment 1, wherein the substrate is at least one of: biodegradable, compostable, or recyclable.
Embodiment 3. The environmental sensor of embodiments 1 or 2, wherein the first layer is formed of a silver ink.
Embodiment 4. The environmental sensor of embodiments 1 through 3, wherein a first electrode in the at least three electrodes is a working electrode, a second electrode in the at least three electrodes is a reference electrode, and a third electrode in the at least three electrodes is a counter electrode.
Embodiment 5. The environmental sensor of embodiments 1 through 4, wherein the working electrode is a gate material comprising a blend of carbon ink and an indicator.
Embodiment 6. The environmental sensor of embodiments 1 through 5, wherein the indicator is alizarin.
Embodiment 7. The environmental sensor of embodiments 1 through 6, wherein the gate material has about 10% weight percentage of alizarin.
Embodiment 8. The environmental sensor of embodiments 1 through 7, wherein the reference electrode is a silver/silver chloride gate.
Embodiment 9. The environmental sensor of embodiments 1 through 8, wherein the semiconductor channel is formed of poly(3,4-ethylenedioxythiopene) polystyrene sulfonate (PEDOT:PSS).
Embodiment 10. The environmental sensor of embodiments 1 through 9, wherein the substrate is formed of polyethylene naphthalate (PEN).
Embodiment 11. A method for gathering data from an environmental sensor, comprising: providing an environmental sensor, wherein the environmental sensor includes a substrate, a silver contact, gate electrodes, a semiconducting channel, and a dielectric encapsulant; implanting the environmental sensor into an environment; determining an electrical conductivity (EC) of the environment; calculating a temperature of the environment; and measuring a pH of the environment.
Embodiment 12. The method of embodiment 11, wherein determining the electrical conductivity includes: interrogating a transistor potentiostatically; and extracting a steady-state current response.
Embodiment 13. The method of embodiments 11 or 12, wherein calculating the temperature includes applying a ratio to the EC and a normalized EC.
Embodiment 14. The method of embodiments 11 through 13, wherein measuring the pH includes: performing a square wave voltammetry (SWV) scan; and comparing a peak voltage to a calibration curve.
Embodiment 15. The method of embodiment 11 through 14, further comprising monitoring a water level of the environment by examining a transistor output characteristic.

What is claimed is:

1. An environmental sensor, comprising:
a substrate, wherein the substrate is reasonably flat;
a first layer affixed to the substrate, the first layer including at least one source electrode and at least one drain electrode;
a second layer affixed to the first layer, the second layer including at least three electrodes;
a third layer affixed to the second layer, the third layer including a semiconducting channel; and
a fourth layer affixed to the third layer, the fourth layer including a dielectric ink.

2. The environmental sensor of claim 1, wherein the substrate is at least one of: biodegradable, compostable, or recyclable.

3. The environmental sensor of claim 1, wherein the first layer is formed of a silver ink.

4. The environmental sensor of claim 1, wherein a first electrode in the at least three electrodes is a working electrode, a second electrode in the at least three electrodes is a reference electrode, and a third electrode in the at least three electrodes is a counter electrode.

5. The environmental sensor of claim 4, wherein the working electrode comprises a gate material comprising a blend of carbon ink and an indicator.

6. The environmental sensor of claim 5, wherein the indicator comprises alizarin.

7. The environmental sensor of claim 6, wherein the gate material comprises about 10% weight percentage of alizarin.

8. The environmental sensor of claim 4, wherein the reference electrode comprises a silver/silver chloride gate.

9. The environmental sensor of claim 1, wherein the semiconducting channel comprises poly(3,4-ethylenedioxythiopene) polystyrene sulfonate (PEDOT:PSS).

10. The environmental sensor of claim 1, wherein the substrate comprises polyethylene naphthalate (PEN).

11. A method for gathering data from an environmental sensor, comprising:
providing the environmental sensor, wherein the environmental sensor includes a substrate, a silver contact, gate electrodes, a semiconducting channel, and a dielectric encapsulant;
implanting the environmental sensor into an environment;
determining, with the environmental sensor, an electrical conductivity (EC) of the environment;
calculating, with the environmental sensor, a temperature of the environment; and
measuring, with the environmental sensor, a pH of the environment.

12. The method of claim 11, wherein determining the electrical conductivity includes:
interrogating a transistor potentiostatically; and
extracting a steady-state current response.

13. The method of claim 11, wherein calculating the temperature includes applying a ratio to the EC and a normalized EC.

14. The method of claim 11, wherein measuring the pH includes:
performing a square wave voltammetry (SWV) scan; and
comparing a peak voltage to a calibration curve.

15. The method of claim 11, further comprising monitoring a water level of the environment by examining a transistor output characteristic.
